(19) United States
(12) Patent Application Publication
(10) Pub. No.: US 2026/0256383 A1
(43) Pub. Date: Sep. 3, 2026

(54) RECOGNIZING FREEZING OF GAIT AND DEPLOYING VIBRATION TO MITIGATE SYMPTOMS

(71) Applicants: Virginia Commonwealth University, Richmond, VA (US); College of William & Mary, Williamsburg, VA (US)

(72) Inventors: Ingrid Pretzer-Aboff, Mechanicsville, VA (US); Leslie Cloud, Henrico, VA (US); Gang Zhou, Williamsburg, VA (US); Kenneth H. Koltermann, Williamsburg, VA (US)

(21) Appl. No.: 18/994,445

(22) PCT Filed: Jul. 19, 2023
(86) PCT No.: PCT/US2023/070478

§ 371 Date: Jan. 14, 2025

Related U.S. Application Data
(60) Provisional application No. 63/391,106, filed on Jul. 21, 2022.

(51) Int. Cl. A61B 5/11; A61B 5/00; G06N 3/0464; G06V 40/20; G16H 50/20

(52) U.S. Cl. CPC A61B 5/112; A61B 5/0051; A61B 5/726; G06N 3/0464; G06V 40/25; G16H 50/20

(57) ABSTRACT

Various embodiments of the present disclosure are directed to systems and methods of utilizing a convolutional neural network to detect freezing of gait in patients and provide vibration to return to normal motor function. A system can perform a method of training a convolutional neural network by receiving a video recording; receiving a plurality of timestamp selections indicating when the person demonstrates a freezing of gait event during the video; receiving movement data from the sensing device; extracting domain data from the movement data; providing the plurality of domain data to the convolutional neural network; and deploying the convolutional neural network on a client device. Additionally, a system can be arranged to perform a method of receiving movement data from the sensing device; determining a categorization of the movement by processing the movement data through a convolutional neural network; and directing a vibration delivery device to deliver vibration.

### CROSS-REFERENCE TO RELATED APPLICATIONS

This application claims priority to, and the benefit of, U.S. Provisional Patent Application 63/391,106, entitled “RECOGNIZING FREEZING OF GAIT AND DEPLOYING VIBRATION TO MITIGATE SYMPTOMS,” which was filed on Jul. 21, 2022, and is incorporated by reference as if set forth herein in its entirety.

### STATEMENT REGARDING FEDERALLY SPONSORED RESEARCH OR DEVELOPMENT

This invention was made with government support under grant number R01 NS120560 awarded by the National Institutes of Health (NIH). The government has certain rights in the invention.

### BACKGROUND

Gait traditionally refers to locomotion achieved through the movement of limbs. In some instances, gait may be directed toward a linear path. In other instances, gait may include turning or similar movements that modify a path of motion during ambulatory activities, such as walking or running.

Parkinson's disease is a chronic, progressive neurodegenerative disease that affects 10 million people worldwide. Parkinson's disease often affects a person's gait. More than half of patients with Parkinson's disease report a gait abnormality called freezing of gait, which is characterized by the periodic inability to initiate or continue normal heel-toe walking. This inability to continue normal heel-toe walking for such a duration can often be incredibly debilitating to patients diagnosed with Parkinson's disease.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is a drawing of a network environment according to various embodiments of the present disclosure.

FIG. 2 is a flowchart illustrating one example of functionality implemented as portions of an application executed in network environment of FIG. 1 according to various embodiments of the present disclosure.

FIG. 3 a flowchart illustrating one example of functionality implemented as portions of an application executed in network environment of FIG. 1 according to various embodiments of the present disclosure.

FIG. 4 is a sequence diagram illustrating one example of the interactions between the components of the network environment of FIG. 1 according to various embodiments of the present disclosure.

### DETAILED DESCRIPTION

Disclosed are various approaches for recognizing freezing of gait and deploying vibration to mitigate the symptoms. Gait traditionally refers to locomotion achieved through the movement of limbs. In some instances, gait can be directed toward a linear path. In other instances, gait can include turning or similar movements that modify a path of motion during ambulatory activities, such as walking or running. Often, patients can present with symptoms affecting their gait, which a physician can diagnose as a variety of diseases or disorders. For example, Parkinsonism is a category of neurological disorders that often affect a patient's gait. At least one example of parkinsonism is Parkinson's disease (“PD”), which is a chronic, progressive neurodegenerative disease that affects 10 million people worldwide. PD is incurable, but treatments exist to reduce the severity of its symptoms. PD and other forms of Parkinsonism can often affect a person's gait. In fact, more than half of patients diagnosed with PD report one or more gait abnormalities. Gait abnormalities can include Hemiplegic gait, Diplegic Gait, Spastic Gait, Neuropathic Gait, Steppage Gait, Equine Gait, Myopathic Gait, Waddling Gait, Choreiform Gait, Hyperkinetic Gait, Ataxic Gait, Parkinsonian Gait, Sensory Gait, Propulsive Gait, and Freezing of Gait (“FoG”). Each of these gait abnormalities can be characterized based on different movement patterns presented by patients.

One such gait abnormality is Freezing of Gait, which is characterized by the periodic inability to initiate or continue normal heel-toe walking. The duration of a FoG episode can range from a fraction of a second up to ten minutes. This inability to continue normal heel-toe walking for such a duration can often be incredibly debilitating to patients diagnosed with PD. Patients exhibiting FOoG often have frequent injurious falls, loss of mobility, decreased social participation, reduced independence, institutionalization, and an overall reduction in quality of life. Prior to an episode of FoG, patients can be seen taking short asynchronous steps known as a pre-FoG gait. FoG episodes are often triggered by various environmental and psychological factors. Some of the most widely recognized triggers are start hesitation (freezing upon gait initiation), walking through tight quarters, turn hesitation (freezing when changing directions), approaching a visual target or destination, dual-tasking, and participating in stressful, time-sensitive situations such as answering the phone or entering an elevator. However, each patient diagnosed with PD can have various, unique triggers for FoG, underscoring a need for customized therapeutic approaches. Although this disclosure primarily discusses effects of and treatments for FoG as an example of a gait abnormality, the systems and methods discussed in this disclosure can also be used to recognize one or more of the previously mentioned gait abnormalities. Additionally, although this disclosure primarily discusses patients affected by PD, the systems and methods discussed in this disclosure can also be used to recognize one or more of the previously mentioned gait abnormalities in patients affected by a variety of diagnoses, such as progressive supranuclear palsy, multiple system atrophy, corticobasal degeneration, vascular parkinsonism, normal pressure hydrocephalus, stroke, cerebral palsy, multiple sclerosis, and various other diagnoses.

Although several pharmacologic agents exist to treat the appendicular and axial motor features of PD, none have demonstrated a benefit for FoG. In fact, many pharmacologic agents can exacerbate FoG in patients. Invasive surgical procedures such as deep brain stimulation can improve certain gait symptoms. However, FoG is frequently resistant to deep brain stimulation. As such, solutions to treat FoG in PD patients are greatly needed.

Many devices used to treat FoG are “open-loop” systems (e.g., delivering a constant/rhythmical stimulus) rather than a more effective “closed-loop” systems (e.g., intermittent stimulus based on a biofeedback paradigm). Open-loop systems can result in habituation, which is a decrease in responsiveness to a stimulus after repeated exposure. As such, closed-loop systems have greater potential to improve long-term FoG outcomes than open-loop options.

One of the most promising options for treating FoG is “cueing,” the introduction of a visual (e.g., laser canes, Google Glass), auditory (e.g., metronomes, electronic device beeps), or tactile (e.g., vibration) stimulus to facilitate the initiation of gait. However, current cueing devices are limited. Some cueing devices require the patient to initiate a cue upon FoG, which can present challenges due to the cognitive issues frequently associated with advanced PD. Auditory and visual cueing are likely ineffective in community settings due to distractions, interruptions, and noise. There are no cueing devices, to date, which provide customized therapy that adapts to the individual needs with regard to the specific triggering scenarios.

Due to the inherent challenges and disappointing outcomes associated with both visual and auditory cueing, vibrational cueing has emerged as a promising and potentially superior alternative. One possible explanation for how vibration therapy works is that vibration triggers an alternative pathway for movement generation in the basal ganglia (BG). Studies suggest that the BG can be bypassed by using external stimuli (e.g., vibration) to prompt other motor regions of the brain, such as the premotor cortex to initiate and maintain movement. Another possible explanation is that the sensory stimulation provided by the vibration could enhance sensorimotor processing, which is deficient in persons with PD. Additionally, a few animal and human experiments found that vibration stimuli influence the concentrations of several neurotransmitters, including biogenic amines that are critical for normal motor function.

As such, various embodiments of the present disclosure are directed to systems and methods of utilizing a convolutional neural network to detect FoG in patients and provide vibration to return to normal motor function. To do this, a system can be arranged to perform a method of training a convolutional neural network by receiving a video recording; receiving a plurality of timestamp selections indicating when the person demonstrates a FoG event during the video; receiving movement data from the sensing device associated with the video recording; extracting a plurality of time domains from the movement data corresponding to the plurality of timestamp selections; sending the plurality of time domains to the convolutional neural network; and deploying the convolutional neural network. Additionally, a system can be arranged to perform a method of receiving movement data from the sensing device for a movement by a patient; determining a categorization of the movement by processing the movement data through a convolutional neural network; and directing a vibration delivery device to deliver vibration in response to determining that the categorization of the movement is indicative of FoG.

In the following discussion, a general description of the system and its components is provided, followed by a discussion of the operation of the same. Although the following discussion provides illustrative examples of the operation of various components of the present disclosure, the use of the following illustrative examples does not exclude other implementations that are consistent with the principles disclosed by the following illustrative examples.

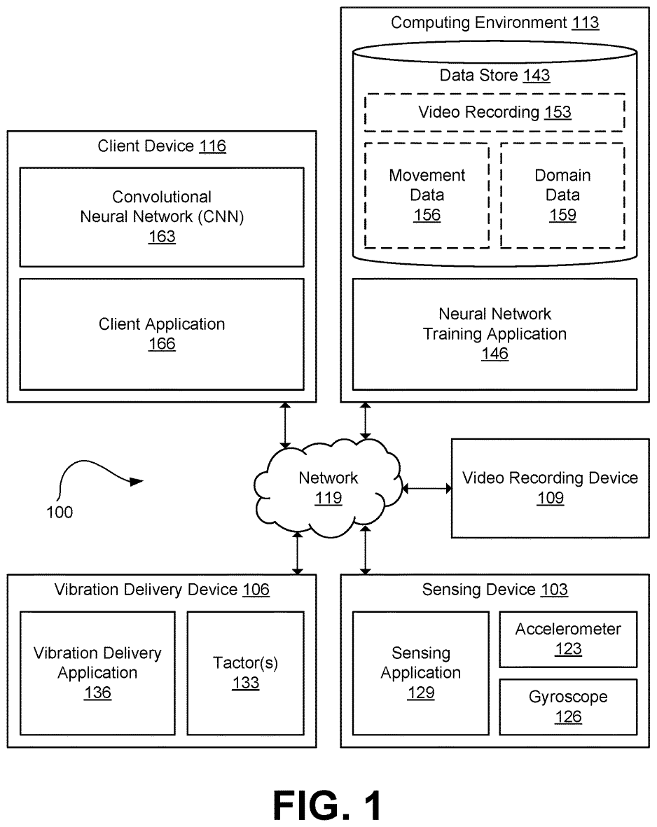

With reference to FIG. 1, shown is a network environment 100 according to various embodiments. The network environment 100 can include a sensing device 103, a vibration delivery device 106, a video recording device 109, a computing environment 113, and a client device 116, which can be in data communication with each other via a network 119.

The network 119 can include wide area networks (WANs), local area networks (LANs), personal area networks (PANs), or a combination thereof. These networks can include wired or wireless components or a combination thereof. Wired networks can include Ethernet networks, cable networks, fiber-optic networks, and telephone networks such as dial-up, digital subscriber line (DSL), and integrated services digital network (ISDN) networks. Wireless networks can include cellular networks, satellite networks, Institute of Electrical and Electronic Engineers (IEEE) 802.11 wireless networks (e.g., WI-FI®), BLUETOOTH® networks, microwave transmission networks, as well as other networks relying on radio broadcasts. The network 119 can also include a combination of two or more networks 119. Examples of networks 119 can include the Internet, intranets, extranets, virtual private networks (VPNs), and similar networks. In at least some embodiments, the sensing device 103 can be connected to the client device 116 over a BLUETOOTH® network. In at least another embodiment, the sensing device 103 can be connected to the client device 116 over a WI-FI® network. In at least some embodiments, the vibration delivery device 106 can also be connected to the client 116 over a BLUETOOTH® network. In at least another embodiment, the sensing device 103 can be connected to the client device 116 over a WI-FI® network.

The sensing device 103 can be one or more computing devices that can be coupled to the network 119. The sensing device 103 can include a processor-based system such as a computer system. Such a computer system can be embodied in the form of a mobile computing device (e.g., personal digital assistants, cellular telephones, smartphones, web pads, tablet computer systems, music players, portable game consoles, electronic book readers, and similar devices), a videogame console, or other devices with like capability. In many embodiments, the sensing device 103 can be a specialized computing device made specifically for collecting movement data of a person. To collect such movement data, the sensing device 103 can include one or more accelerometers 123 and/or one or more gyroscopes 126 to measure movement data for a person.

In many embodiments, the sensing device 103 can be worn on a specific body part to collect body part-specific movement data. The sensing device 103 can be worn on various parts of the body. In many embodiments, the sensing device 103 can be worn on a person's leg (e.g., the ankle, the calf, the knee joint, and/or the thigh). In some embodiments, the sensing device 103 can be worn around other parts of the body (e.g., the waist, the arm, the chest, the neck, the abdomen). In at least some embodiments, a person can wear more than one sensing device 103 over various parts of the body. However, in many embodiments, a person can wear two sensing devices 103, a first sensing device 103 on a first ankle and a second sensing device 103 on a second ankle. In such an embodiment, the sensing devices 103 can detect movement from each leg, which can provide more reliable movement data to detect FoG.

The sensing device 103 can include one or more accelerometers 123. Each accelerometer 123 can detect a magnitude and direction of the acceleration of the sensing device 103 as it is being moved. The accelerometers 123 can be single-axis or multi-axis accelerometers 123. A single-axis accelerometer 123 can provide a single value corresponding to a specified axis to the sensing application 129. Using more than one single-axis accelerometers 123 individually collecting data over multiple axes' can yield the magnitudes for each axis. Alternatively, the sensing device 103 can use one multi-axis accelerometer 123 to yield the same results. In many embodiments, the one or more accelerometers 123 can detect the magnitude of the movement with very great precision. In at least one embodiment, a 3-axis accelerometer 123 is capable of recording the magnitude of movement at 100 Hz.

The sensing device 103 can include one or more gyroscopes 126. Each gyroscope 126 can detect an angular velocity of the sensing device 103 as it is being moved and/or rotated. The gyroscopes 126 can be single-axis or multi-axis gyroscopes 126. A single-axis gyroscope 126 can provide a single value corresponding to a specified axis to the sensing application 129. Using more than one single-axis gyroscope 126 individually collecting data over multiple axes' can yield the angular velocity for each axis. Alternatively, the sensing device 103 can use one multi-axis gyroscope 126 to yield the same results. In many embodiments, the one or more gyroscope 126 can detect the angular velocity with very great precision. In at least one embodiment, a 3-axis gyroscope 126 is capable of recording the angular velocity at 100 Hz.

The sensing device 103 can be configured to execute various applications such as a sensing application 129, or other applications. The sensing device 103 can also be configured to execute applications beyond the sensing application 129, if necessary. The sensing application 129 can be configured to collect, obtain, and/or receive data corresponding to magnitude of a movement detected by one or more accelerometers 123. The sensing application 129 can be configured to collect, obtain, and/or receive data corresponding to angular velocity of a movement detected by one or more gyroscopes 126. The sensing application 129 can combine the collected, obtained, and/or received data from the accelerometers 123 and the gyroscopes 126 to generate movement data that can be sent to other devices and/or other applications. In at least one embodiment, the sensing application 129 can transmit the movement data over the network 119 to the neural network training application 146 of the computing environment 113. In at least another embodiment, the sensing application 129 can transmit the movement data over the network 119 to the client device 116. In at least one embodiment, the sensing application 129 can send the movement data to the client device 116 in real-time as the movement data is obtained.

The vibration delivery device 106 can be one or more computing devices that can be coupled to the network 119. The vibration delivery device 106 can include a processor-based system, such as a computer system. In at least one embodiment, the vibration delivery device 106 can be embodied in the form of a mobile computing device (e.g., personal digital assistants, cellular telephones, smartphones, web pads, tablet computer systems, music players, portable game consoles, electronic book readers, and similar devices), a videogame console, or other devices with at least a processor, a network device to connect to a network 119, and/or one or more devices capable of delivering vibration, rumble, or haptic feedback. In at least another embodiment, the vibration delivery device 106 can be a specialized computing device capable of delivering vibration to a body part in response to receiving a signal over the network 119. In at least one embodiment, the received signal can indicate that vibration simply needs to be delivered to the wearer. In such an embodiment, the vibration delivery device 106 can decide what strength, pattern, and duration of vibration to provide. In at least another embodiment, the received signal can specifically dictate the strength, pattern, and duration of vibration to provide.

To generate such vibration, the vibration delivery device 106 can include one or more tactors 133. A tactor 133 can be a device that is capable of generating vibration. In at least one embodiment, the one or more tactors 133 can contain one or more linear actuators with a moving magnet design that can generate a vibrational frequency between 5 Hz and 400 Hz having a max peak to peak displacement (amplitude) between 0.2 mm and 2 mm when loaded. In at least one embodiment, the one or more tactors 133 can generate a vibrational frequency of 300 Hz and having a max peak to peak displacement (amplitude) of 0.8 mm when loaded. Each of the one or more tactors 133 can generate vibration independently of each other, but concurrent vibration from one or more of the one or more tactors 133 can also occur. The tactors 133 can generate vibration to be sustained for any period of time. In at least one embodiment, the tactors 133 can provide a sustained amount of vibration for a period of time between 0.5 seconds and 5 seconds. In at least another embodiment, the tactors 133 can provide a longer sustained vibration for a period between 5 seconds and 10 seconds.

The one or more tactors 133 can be attached to the outside of a human body. In at least one embodiment, the vibration delivery device 106 can include two tactors 133. In such an embodiment, a first tactor 133 can be worn on or applied to the medial aspect of the malleolus, the bony prominence on the inner side of the ankle formed by the lower end of the fibula. A second tactor 133 can be worn on or applied to the dorsal surface of the foot, the surface facing upwards while standing.

The vibration delivery device 106 can be configured to execute various applications, such as a vibration delivery application 136 or other applications. The vibration delivery device 106 can also be configured to execute applications beyond the vibration delivery application 136, if necessary. The vibration delivery application 136 can be configured to receive signals from the network 119 indicating that vibrations need to be delivered to the wearer of the vibration delivery device 106. In such an embodiment, the vibration delivery application 136 can decide what strength, pattern, and duration of vibration to provide. In at least another embodiment, vibration delivery application 136 can receive a signal which specifically dictates the strength, pattern, and duration of vibration to provide. The vibration delivery application 136 can direct the one or more tactors 133 to generate vibration. The vibration delivery application 136 can direct each of the one or more tactors 133 individually or in unison. The vibration delivery application 136 can direct the one or more tactors 133 to generate the vibration at various times, in various strengths (amplitude), frequencies, patterns, and durations. The vibration delivery application 136 can direct the tactors 133 to deliver vibrations at a sustained frequency and a sustained amplitude for a period of time, like delivering a vibration at 240 Hz at an amplitude of 0.6 millimeters for 2 seconds. The vibration delivery application 136 can also direct the tactors 133 to deliver vibrations at variable frequencies, such as sinusoidal amounts of frequency over time which makes it feel like the vibrational frequency is changing, at a sustained amplitude or strength. The vibration delivery application 136 can also direct the tactors 133 to deliver vibrations at sustained frequencies, but at variable amplitudes, such as a sinusoidal amount of amplitude over a period of time to make the same vibration frequency making the strength of the vibration feel different. Various combinations of sustained and variable frequencies, strengths (amplitude), patterns, and times can be combined.

The vibration delivery application 136 can correlate each of the one or more tactors 133 with a specific location of the human body. For example, the vibration delivery application 136 can correlate a first tactor 133 as being worn on or applied to the medial aspect of the malleolus and a second tactor 133 as being worn on or applied to the dorsal surface of the foot. Using such correlation, the vibration delivery application 136 can receive a signal from the network 119 that indicates that the medial aspect of the malleolus should receive vibration and the vibration delivery application 136 can direct the first tactor 133 to generate such vibration.

The vibration delivery device 106 can also be physically combined with the sensing device 103 as a single wearable device. Combining the vibration delivery device 106 and the sensing device 103 can be beneficial to a patient because fewer devices are required to be worn. Additionally, when the vibration delivery device 106 and the sensing device 103 are combined and worn by a patient in a consistent location on the body (e.g. a foot, an ankle, etc.), applications on the computing device 112 or the mobile device 115 can better recognize patterns of movement for a patient over various days of wearing the combined vibration delivery device 106 and sensing device 103. As a result, there should be fewer mistakes when detecting FoG episodes for patients consistently wearing the combined vibration delivery device 106 and sensing device 103.

The video recording device 109 can be any device capable of capturing video recordings. For instance, the video recording device 109 can be a camcorder (digital or analog), a digital camera capable of capturing video, a mobile computing device capable of capturing video (e.g., personal digital assistants, cellular telephones, smartphones, web pads, tablet computer systems, music players, portable game consoles, electronic book readers, and similar devices), a videogame console, or other like devices capable of capturing video. In at least one embodiment, the video recording device 109 can include one or more computing devices that include a processor, a memory, and/or a network interface. In at least another embodiment, the video recording device 109 can be an analog video camera.

The video recording device 109 can be used by a physician, a nurse, a certified medical professional, a medical technician, a staff member, a patient, or any person to capture a video of a patient walking through a predetermined course. The predetermined course can include obstacles for the patient that are common triggers of FoG, such as various turns, various pathways, various uneven terrain, etc. While the patient is walking through the predetermined course, the patient can also wear the sensing device 103. The sensing device 103 can generate movement data for the patient as they walk through the predetermined course. Once the patient has completed the predetermined course, the sensing device 103 can send the computing environment 113 the movement data and the video recording device 109 can send the video of the patient to the computing environment 113. In some embodiments, the video recording device 109 is an analog video recorder and it may not have the capability to send the video to the computing environment 113. In such a situation, the analog video recording can be digitized and provided to the computing environment 113.

The computing environment 113 can include one or more computing devices that include a processor, a memory, and/or a network interface. For example, the computing devices can be configured to perform computations on behalf of other computing devices or applications. As another example, such computing devices can host and/or provide content to other computing devices in response to requests for content. Moreover, the computing environment 113 can employ a plurality of computing devices that can be arranged in one or more server banks or computer banks or other arrangements. Such computing devices can be located in a single installation or can be distributed among many different geographical locations. For example, the computing environment 113 can include a plurality of computing devices that together can include a hosted computing resource, a grid computing resource, or any other distributed computing arrangement. In some cases, the computing environment 113 can correspond to an elastic computing resource where the allotted capacity of processing, network, storage, or other computing-related resources can vary over time.

Alternatively, the computing environment 113 can be one or more computing devices that can be coupled to the network 119. The computing environment 113 can include a processor-based system such as a computer system. Such a computer system can be embodied in the form of a personal computer (e.g., a desktop computer, a laptop computer, or similar device), a mobile computing device (e.g., personal digital assistants, cellular telephones, smartphones, web pads, tablet computer systems, music players, portable game consoles, electronic book readers, and similar devices), a videogame console, or other devices with like capability. The computing environment 113 can include one or more displays, such as liquid crystal displays (LCDs), gas plasma-based flat panel displays, organic light emitting diode (OLED) displays, electrophoretic ink (“E-ink”) displays, projectors, or other types of display devices. In some instances, the display can be a component of the computing environment 113 or can be connected to the computing environment 113 through a wired or wireless connection.

In many embodiments, the computing environment 113 can have a data store 143. The data store 143 can be representative of a plurality of data stores 143, which can include relational databases or non-relational databases such as object-oriented databases, hierarchical databases, hash tables or similar key-value data stores, as well as other data storage applications or data structures. Moreover, combinations of these databases, data storage applications, and/or data structures may be used together to provide a single, logical, data store. Various data can be stored in the data store 143 that is accessible to the computing environment 113. The data stored in the data store 143 is associated with the operation of the various applications or functional entities described below. This data can include video recordings 153, movement data 156, domain data 159, and potentially other data.

The video recordings 153 can represent one or more videos recorded by one or more video recording devices 109. The video recordings 153 can include video and audio depictions of a patient walking through a predetermined course. The predetermined course can include obstacles for the patient that are common triggers of FoG, such as various turns, various pathways, various uneven terrain, etc. The predetermined course can be set up in the physical world, typically at a physician's office, hospital, or a medical testing facility. Alternatively, the predetermined course can be set up in the virtual world, accessed via a virtual reality (VR) headset or augmented reality (AR) headset. While the patient is walking through the predetermined course in the video recordings 153, the patient can also be wearing the sensing device 103. The sensing device 103 depicted in the video recording 153 can generate movement data 156 for the duration of the video. Once the patient completed the predetermined course, the video recording 153 can end. The computing environment 113 can receive the video recording 153 from the video recording device 109 or from other sources.

A physician and/or certified medical professional can watch the video recording 153 to categorize each movement of the patient. For instance, a physician can watch a portion of a patient's step in the video recording 153 and determine that the movement corresponded to a standard walking pattern. A physician can also watch another portion of the video recording 153 to determine that the patient is beginning to exhibit symptoms related to the start of a FoG episode. The physician can watch yet another portion of the video recording 153 to determine that the patient is exhibiting a FoG episode. The physician can categorize each of these movements in various ways, such as first contact of a footstep, last contact of a footstep, intentional stop, unintentional stop (FoG), walking patterns associated with the start of a FoG episode, turns, and other types of categorizations for the patient's movement. These movement categorizations corresponding to the times in which the patient performed certain movements in the predetermined course, can be used in conjunction with the movement data 156 from the sensing device 103 to train a convolutional neural network using the neural network training application 146. Although the previous example stated a physician, it should be understood that a certified medical professional could also perform the categorization as stated in the example.

The movement data 156 can represent the data collected by a sensing device 103 while the patient is walking through the predetermined course during a video recording 153. In some embodiments, the movement data 156 can include at least one of the magnitudes of a movement detected by one or more accelerometers 123 and/or the angular velocity of a movement detected by one or more gyroscopes 126. In at least one embodiment, the movement data 156 can at least include measurements collected from a 3-axis accelerometer 123. The movement data 156 can also include measurements collected from a 3-axis gyroscope 126. Other data could also be included in the movement data 156, such as relative location in the predetermined course, or other important information in determining the movement of a patient.

The domain data 159 can represent time domains and/or frequency domains of the movement data 156. A time domain of the movement data 156 is a discreet segment of the movement data 156 that is extrapolated based on a time graph of the data. A dataset, such as the movement data 156, can be extrapolated to generate a plurality of time domains. These time domains can be normalized A frequency domain of the movement data 156 is a discrete segment of the movement data 156 that is extrapolated based on a frequency graph of the data. In at least one embodiment, the frequency domains can be generated by performing continuous wavelet transformation (CWT) over the time domains. In at least another embodiment, the frequency domains can be generated by applying bandpass filters to the time domain data. In at least another embodiment, the frequency domains can be generated by applying Fast Fourier Transform (FFT) over the time domains. Frequency domains can be important for a convolutional neural network to recognize dominant frequencies for normal walking as compared to dominant frequencies from FoG events. Uses of the domain data 159 will be further explained in the discussion of the neural network training application 146.

Traditionally, before training neural networks, the domain data 159 can be split into training, validation, and testing sets. The training domain data 159 is used to fit a scaling algorithm, which is then used to transform the training, validation and testing sets to some arbitrary scale (typically 0 to 1). However, neural networks trained with normalized domain data 159 typically converge faster and produce better results than models trained with raw unnormalized data, often not needing extensive validation and testing sets. Although some embodiments can normalize a specific patient's domain data 159 with the domain data 159 of various patients, many embodiments normalize each patient's domain data 159 individually among itself. When a patient's domain data 159 is normalized individually, the amplitude and frequency of a patient's normal gait become more pronounced and any deviations from the normal gait also become more pronounced. As such, in various embodiments, each facet of the domain data 159 (time domains, frequency domains, etc.) can be normalized.

The computing environment 113 can be configured to execute various applications, such as a neural network training application 146 or other applications. The neural network training application 146 can be executed in a computing environment 113 to access network content served up on the network 119, thereby rendering a user interface on the display. To this end, neural network training application 146 can include a browser, a dedicated application, or other executables, and the user interface can include a network page, an application screen, or other user mechanisms for obtaining user input. The computing environment 113 can be configured to execute applications beyond the neural network training application 146, such as email applications, social networking applications, word processors, spreadsheets, or other applications.

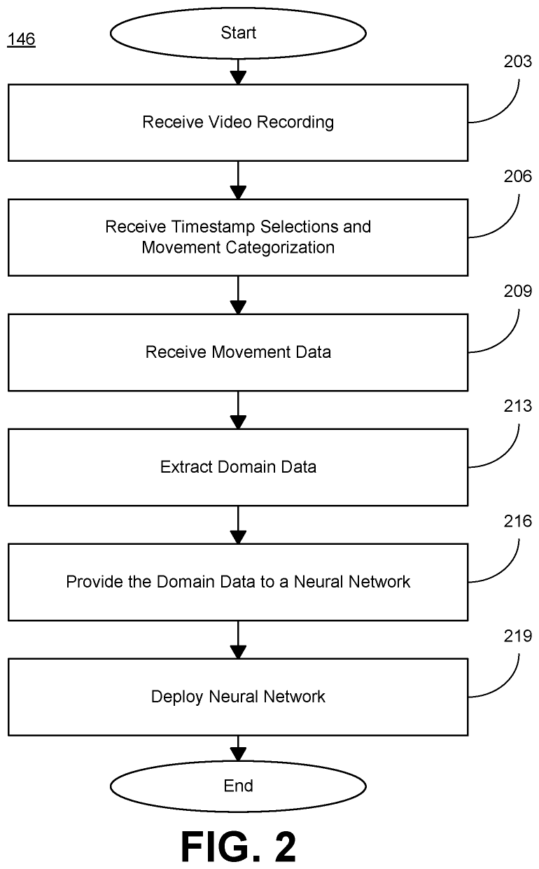

The neural network training application 146 can be executed to receive movement data 156 and video recordings 153 associated with a patient moving through a predetermined course, process the patient's movement data 156 into domain data 159, train a convolutional neural network, and deploy the convolutional neural network. First, the neural network training application 146 can receive a video recording 153 associated with a patient moving through a predetermined course. Next, the neural network training application 146 can receive timestamp selections that correspond to various categorizations of movements represented in the video recording 153, such as first contact of a footstep, last contact of a footstep, intentional stop, unintentional stop (FoG), walking patterns associated with the start of a FoG episode, turns, and other types of categorizations for the patient's movement. Next, the neural network training application 146 can receive the movement data 156. Next, the neural network training application 146 can extract the domain data 159 from the movement data 156 based on the timestamp selections corresponding to movements represented in the video recording 153. The extracted domain data 159 can include time domain data and/or frequency domain data. Next, the neural network training application 146 can train the convolutional neural network using the domain data 159. Finally, the neural network training application 146 can deploy the convolutional neural network. This process is further explained in the discussion of FIG. 2.

The client device 116 is representative of a plurality of client devices that can be coupled to the network 119. The client device 116 can include a processor-based system such as a computer system. Such a computer system can be embodied in the form of a personal computer (e.g., a desktop computer, a laptop computer, or similar device), a mobile computing device (e.g., personal digital assistants, cellular telephones, smartphones, web pads, tablet computer systems, music players, portable game consoles, electronic book readers, and similar devices), media playback devices (e.g., media streaming devices, BluRay® players, digital video disc (DVD) players, set-top boxes, and similar devices), a videogame console, or other devices with like capability. The client device 116 can include one or more displays, such as liquid crystal displays (LCDs), gas plasma-based flat panel displays, organic light emitting diode (OLED) displays, electrophoretic ink (“E-ink”) displays, projectors, or other types of display devices. In some instances, the display can be a component of the client device 116 or can be connected to the client device 116 through a wired or wireless connection.

The client device 116 can be configured to execute various applications such as a convolutional neural network (CNN) 163, a client application 166, or other applications. The client device 116 can be configured to execute applications beyond the client application 166, such as email applications, social networking applications, word processors, spreadsheets, or other applications.

The client device 116 can execute the CNN 163. The CNN 163 can be executed to, after being trained to do so, categorize movement based on real-time movement data sent from a sensing device 103 being worn by a patient. As such, the CNN 163 can detect FoG episodes in a patient in real-time. The CNN 163 can be trained to categorize movement data 156 in the computing environment 113 and then subsequently deployed on the client device 116 for use by the patient. Although the CNN 163 can exist in various different embodiments, there are three common models for which the CNN 163 adheres: a time domain CNN model, a time domain and Butterworth frequency domain CNN model, and a time domain and CWT frequency domain CNN model. These three models can differ by structure and inputs, so the accuracy of the FoG detection and computation speed can be affected based on which model is chosen. Accuracy of the detection of FoG episodes can be traded for computation speed, as needed. For example, speed can be preferable detection accuracy for less severe PD patients who display only a few types of FoG episodes. A more complex and accurate CNN model can be utilized for slower moving PD patients that experience various types of FoG episodes.

The smallest and subsequently fastest model for which a CNN 163 adheres is the time domain CNN model. The time domain CNN model is trained using only time domains of the domain data 159. The time domain CNN model is often faster than other CNN models, but it often comes at the expense of accuracy. The time domains can be inputs to one block of a three-layer, two-dimensional CNN 163 (32 filters, kernel size=(4×3)) with a MaxPooling(pool size=2,1)) layer in between each convolutional layer. The output of the final MaxPooling layer can be flattened and acts as input to the fully-connected layers of our model. Each MaxPooling layer can use a pool size of (2,1) so as to reduce the time domain. Due to these efficiencies, the time domain CNN model can often produce a prediction between 30 milliseconds and 55 milliseconds.

Patients experiencing more severe PD with a wide variety of FoG triggers can often be better treated using a time domain and Butterworth frequency domain CNN model. Although the time domain and Butterworth frequency domain CNN model might be slower than the time domain CNN because it must extrapolate the real-time movement data using Butterworth filters, the accuracy of predicting a FoG event is roughly 4% greater when compared to the time domain CNN model. The time domains can be inputs to three blocks of three-layer, two-dimensional CNN 163 (32 filters, kernel size=(4×3)) with a MaxPooling(pool size=2,1)) layer in between each convolutional layer. The output of the final MaxPooling layer can be flattened and acts as input to the fully-connected layers of this model. Each MaxPooling layer uses a pool size of (2,1). Each of the three blocks of the time domain and Butterworth frequency domain CNN model can learn feature maps for the time domain data, the frequency data filtered to 0.5-3 Hz, and the frequency data filtered to 3-8 Hz, separately. Because of the processing time required, the time domain and Butterworth frequency domain CNN model can often produce a prediction between 150 milliseconds and 175 milliseconds.

Patients experiencing more severe PD with a wide variety of FoG triggers can also be better treated using a time domain and CWT frequency domain CNN model. The time domain and CWT frequency domain CNN model might be slower than the time domain and Butterworth frequency domain CNN, the accuracy of predicting a FoG event is significantly increased when compared to the previously discussed CNN models. The time domains can be inputs to a first block of three-layer, two-dimensional CNN 163 (32 filters, kernel size=(4×3)) with a MaxPooling(pool size=2,1)) layer in between each convolutional layer of this first block. The frequency domains calculated by performing CWT over the time domains can be inputs to a second block of three-layer, two-dimensional CNN 163 (32 filters, kernel size=(4×3)) with a MaxPooling(pool size=2,1)) layer in between each convolutional layer of this second block. Because of the processing time required to process the time domains through a CWT, this model can often take a more time than the previous models.

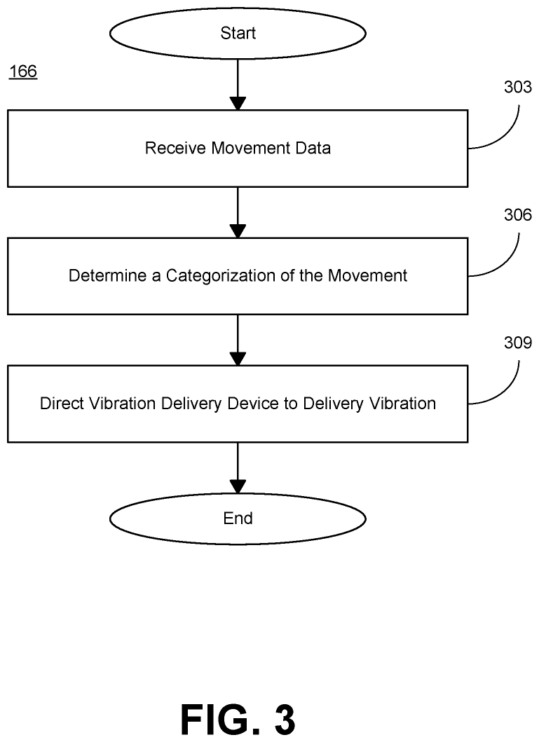

The client device 116 can also execute the client application 166. The client application 166 can be executed to receive real-time movement data from a sensing device 103, determine the categorizations of movement from the real-time movement data collected from a sensing device 103, and then directing a vibration delivery device 106 to deliver vibration to the patient. Further details of the previously mentioned process can be found in the discussion for FIG. 3. Additionally, the client application 166 can also assist in deploying the CNN 163 on the client device 116.

Next, a general description of the operation of the various components of the network environment 100 is provided. To begin, a patient having PD that experiences FoG might be directed by a physician to wear one or more sensing devices 103 while moving through a predetermined course. The predetermined course can include obstacles for the patient that are common triggers of FoG, such as various turns, various pathways, various uneven terrain, etc. While the patient is moving through the predetermined course, a video recording device 109 can capture a video recording 153 of the patient moving throughout the predetermined course while wearing one or more sensing devices 103.

Once the patient has completed the predetermined course, the neural network training application 146 can receive the video recording 153 from the video recording device 109 and the movement data 156 from sensing application 129 on the sensing device 103. The neural network training application 146 can store both the video recording 153 and the movement data 156 in the data store 143. A physician and/or certified medical professional can watch the video recording 153 to categorize each movement of the patient and correspond those movements to timestamps in the video. The physician and/or certified medical professional can categorize each of these movements in various ways, such as first contact of a footstep, last contact of a footstep, intentional stop, unintentional stop (FoG), walking patterns associated with the start of a FoG episode, turns, and other types of categorizations for the patient's movement. The movement categorizations and corresponding timestamps for such movements can be received by the neural network training application 146 on the computing environment 113. The neural network training application 146 can then extract the domain data 159 from the movement data 156 based on the received time stamp selections and movement categorizations. The neural network training application 146 can then train a CNN 163 with the extracted domain data 159. The CNN 163 can subsequently be deployed to a client device 116 for use by a patient. This process is further explained in the discussion of FIG. 2.

Once the CNN 163 has been deployed on the client device 116, the client application 166 can be executed. The client application 166 can receive real-time movement data from one or more sensing devices 103 worn by the patient. The client application 166 can determine a categorization for a movement based on the received real-time movement data from one or more sensing devices 103. The client application 166 can group portions of the real-time movement data together and provide that group of the real-time movement data to the deployed CNN 163 to determine the categorization of the patient's current movements. The client application 166 can receive a response from that CNN 163 that the patient is experiencing or about to begin experiencing a FoG episode. The client application 166 can direct the vibration delivery application 136 of the vibration delivery device 106 to generate vibration for the patient. This process is further explained in the discussion of FIG. 3.

Referring next to FIG. 2, shown is a flowchart that provides one example of the operation of the neural network training application 146. The flowchart of FIG. 2 provides merely an example of the many different types of functional arrangements that can be employed to implement the operation of the depicted portion of the neural network training application 146. Alternatively, the flowchart of FIG. 2 could be viewed as depicting a method implemented by the computing environment 113.

Beginning with block 203, the neural network training application 146 can receive a video recording 153. The neural network training application 146 can receive such a video recording 153 from the video recording device 109. In some embodiments, the video recording device 109 can be an analog video recording device 109, which may not have the capability of sending the video to the computing environment 113. In such a situation, the analog video recording can be digitized into a video recording 153 and provided to the neural network training application 146. The neural network training application 146 can store the video recording 153 in the data store 143 for long-term storage, if necessary.

At block 206, the neural network training application 146 can receive timestamp selections and movement categorizations for the movements in the video recording 153. A physician can watch the video recording 153 to categorize each movement of the patient. For instance, a physician can watch a portion of a patient's step in the video recording 153 and determine that the movement corresponded to a standard walking pattern. A physician can watch another portion of the video recording 153 to determine that the patient is beginning to exhibit symptoms related to the start of a FoG episode. The physician can watch yet another portion of the video recording 153 to determine that the patient is exhibiting a FoG episode. The physician can categorize each of these movements in various ways, such as first contact of a footstep, last contact of a footstep, intentional stop, unintentional stop (FoG), walking patterns associated with the start of a FoG episode, turns, and other types of categorizations for the patient's movement.

These movement categorizations are then attributed to timestamps of the video recording 153. In at least one embodiment, a physician can denote a first timestamp for when a patient begins experiencing a FoG episode. In that same instance, the physician can denote a second timestamp for when the patient ends the FoG episode and returns to a normal walking pattern. Based on these two timestamps, the neural network training application 146 can infer that the FoG episode occurred between the first timestamp and the second timestamp. In at least another embodiment, the physician can explicitly mark a start and end time for each movement categorization. In at least one embodiment, the neural network training application 146 can receive the timestamp selections and movement categorizations from a user of the computing environment. In at least another embodiment, the neural network training application 146 can receive the timestamp selections and movement categorizations from a client device 116. In at least another embodiment, the neural network training application 146 can receive the timestamp selections and movement categorizations from a computing device on the network 119 for which a physician can review the video recording 153 and analyze the movements of the patient. Although the discussion of block 206 repeatedly stated a physician, it should be understood that a certified medical professional could also perform such categorizations.

At block 209, the neural network training application 146 can receive movement data 156 from the sensing application 129 of the sensing device 103. The sensing application 129 can be configured to collect, obtain, and/or receive data corresponding to the magnitude of a movement detected by one or more accelerometers 123, and the angular velocity of a movement detected by one or more gyroscopes 126. The sensing application 129 can combine the collected, obtained, and/or received data from the accelerometers 123 and the gyroscopes 126 to generate movement data 156 that can be sent to the neural network training application 146 of the computing environment 113. The movement data 156 can include the data collected from the accelerometers 123 and/or the gyroscopes 126 while the patient is wearing the sensing device 103 during the patient's walk through the predetermined course, for which the video recording device 109 captured a video recording 153. Stated differently, the movements represented by the movement data 156 correspond to the movements captured in the video recording 153. The movement data 156 can be stored in the data store 143 for long-term storage.

At block 213, the neural network training application 146 can extract domain data 159. The domain data 159 can represent time domains and/or frequency domains of the movement data 156. A time domain of the movement data 156 is a discreet segment of the movement data 156 that is extrapolated based on a time graph of the data. A dataset, such as the movement data 156, can be extrapolated to generate a plurality of time domains. A frequency domain of the movement data 156 is a discrete segment of the movement data 156 that is extrapolated based on a frequency graph of the data. Frequency domains can be important for a convolutional neural network to recognize dominant frequencies for normal walking as compared to dominant frequencies from FoG events.

Because the movements represented by the movement data 156 correspond to the movements captured in the video recording 153, the movement data 156 and the video recording 153 can be synchronized based on time. Once the video recording 153 and the movement data 156 have been synchronized, the timestamps received at block 206 can be used to identify sections of the movement data 156 that correspond to certain categories of movement. Using that information, the neural network training application 146 can extract time domains from the movement data 156 based on the time stamp selections received in block 206. These time domains can be normalized as previously explained in the discussion of domain data 159 in FIG. 1. The time domains can be stored in the data store 143 as domain data 159.

Additionally, the neural network training application 146 can extract frequency domains from the time domains. In at least one embodiment, the frequency domains can be generated by performing continuous wavelet transformation (CWT) over the time domains. In at least another embodiment, the frequency domains can be generated by applying bandpass filters to the time domain data. The frequency domains can also be normalized as previously explained in the discussion of domain data 159 in FIG. 1. The frequency domains can also be stored in the data store 143 as domain data 159.

In certain embodiments, ranges of frequencies can be stored separately. For instance, the frequency domains can be grouped by grouped into two groups of frequencies—low frequencies and high frequencies. In at least one embodiment, low frequencies can be grouped for frequencies between 0.5-3 Hz, and high frequencies can be grouped for frequencies between 3-8 Hz. Groupings of frequencies can be stored in various different arrangements as domain data 159 in the data store 143.

At block 216, the neural network training application 146 can provide the domain data 159 to a neural network. The neural network training application 146 can identify a neural network to train. The neural network training application 146 can identify a neural network on the computing environment 113 or a neural network on any device connected over the network 119. For example, neural network training application 146 can train the CNN 163 on the client device 116 over the network 119. In another example, the neural network training application 146 can generate a new neural network and train this new neural network with the data.

In general, the neural network can adhere to the specifications previously explained in the discussion of common models for the CNN 163 in FIG. 1, such as: a time domain CNN model, a time domain and Butterworth frequency domain CNN model, and a time domain and CWT frequency domain CNN model. When the neural network is already created as a CNN 163 on the client device 116, the neural network training application 146 can identify which model for which the CNN 163 adheres. When the neural network is newly generated on the neural network training application 146, the neural network training application 146 can determine which neural network model is best for the patient by evaluating the severity of the FoG episodes in the movement data 156, the quantity of the FoG episodes in the movement data 156, the speed at which the person walks (as measured in the movement data 156), or other factors that can be gleaned from evaluating the movement data 156. In at least another embodiment, the computing environment 113 can receive input from a user (physician, nurse, certified medical professional, medical technician, medical assistant, data scientist, any person, etc.) indicating which neural network model can be generated. Using that input or the determination from the neural network training application 146 based on the movement data 156, the neural network training application 146 can be generated if it does not already exist.

The neural network training application 146 can begin training the neural network with the domain data 159. The specific type(s) of domain data 159 provided to the neural network with be determined based on the type of neural network model that was identified or chosen. For instance, in a time domain CNN model, the time domains of the domain data 159 can be provided to the neural network, but not the frequency data. In another instance, for a time domain and Butterworth frequency domain CNN model, the time domains of the domain data 159, the frequency domains filtered or grouped to 0.5-3 Hz, and the frequency domains filtered or grouped to 3-8 Hz can each be provided separately to the neural network by the neural network training application 146. In yet another instance, for a time domain and CWT frequency domain CNN model, the time domains of the domain data 159, and the frequency domains (as generated by performing CWT over the time domains of the domain data 159) can each be provided separately to the neural network by the neural network training application 146.

At block 219, the neural network training application 146 can be deployed on the client device 116. In embodiments where the neural network was a pre-existing CNN 163 on the client device 116, deployment can include notifying the client application 166 that the CNN 163 has been trained with new data. In embodiments where the neural network training application 146 has generated a new neural network, the neural network training application 146 can send the neural network to the client application 166 to execute on the client device 116 as the CNN 163. In yet another embodiment, the neural network training application 146 can deploy the neural network as the CNN 163 directly on the client device 116, without involving the client application 166. After the CNN 163 has been deployed on the client device 116, the flowchart of FIG. 2 comes to an end.

Referring next to FIG. 3, shown is a flowchart that provides one example of the operation of the client application 166. The flowchart of FIG. 3 provides merely an example of the many different types of functional arrangements that can be employed to implement the operation of the depicted portion of the client application 166. Alternatively, the flowchart of FIG. 3 could be viewed as depicting a method implemented by the client device 116.

Beginning with block 303, the client application 166 can receive real-time movement data from the sensing application 129 of the sensing device 103. The sensing application 129 can be configured to collect, obtain, and/or receive data corresponding to the magnitude of a movement detected by one or more accelerometers 123 and the angular velocity of a movement detected by one or more gyroscopes 126. The sensing application 129 can combine the collected, obtained, and/or received data from the accelerometers 123 and the gyroscopes 126 to generate real-time movement data that can be sent to the neural network training application 146 of the computing environment 113. The real-time movement data can include the data collected from the accelerometers 123 and/or the gyroscopes 126 while the patient is wearing the sensing device 103.

At block 306, the client application 166 can determine a categorization of the movement represented in the real-time movement data. To do so, the client application 166 can provide the real-time movement data to the CNN 163. The CNN 163 can process the real-time movement data and return a categorization of movement. For instance, the CNN 163 can categorize each of these movements in various ways, such as first contact of a footstep, last contact of a footstep, intentional stop, unintentional stop (FoG), walking patterns associated with the start of a FoG episode, turns, and other possible categories of movement. In at least one embodiment, the CNN 163 simply categorizes the movement as a “start of a FoG episode” or “not the start of a FoG episode.” Before providing the real-time movement data to the CNN 163, the client application 166 can collect a plurality of real-time movement data packets for a specified period of time. In that sense, the CNN can recognize a trend of the movement to better predict whether movement indicates a start of FoG or FoG episode. The client application 166 can continue categorizing real-time movement data until it determines that a start of FoG or FoG episode is occurring, at which point the process moves to block 309.

At block 309, the client application 166 can direct the vibration delivery application 136 of the vibration delivery device 106 to provide the patient with vibration. The client application 166 can receive a categorization of the real-time movement data indicating that the patient is experiencing symptoms of the start of FoG or a FoG episode. As such, the client application 166 can direct the vibration delivery application 136 to deliver vibration to the patient. In at least one embodiment, the client application 166 dictates the strength, frequency, pattern, and duration of vibration for the vibration delivery application 136 to provide to the patient. In at least another embodiment, the client application 166 can direct the vibration delivery application 136 to deliver the vibration and the vibration delivery application 136 dictates the strength, frequency, pattern, and duration of vibration provided to the patient. After the client application 166 has directed the vibration delivery application 136 on the vibration delivery device 106 to deliver vibration to the patient, the flowchart of FIG. 3 comes to an end.

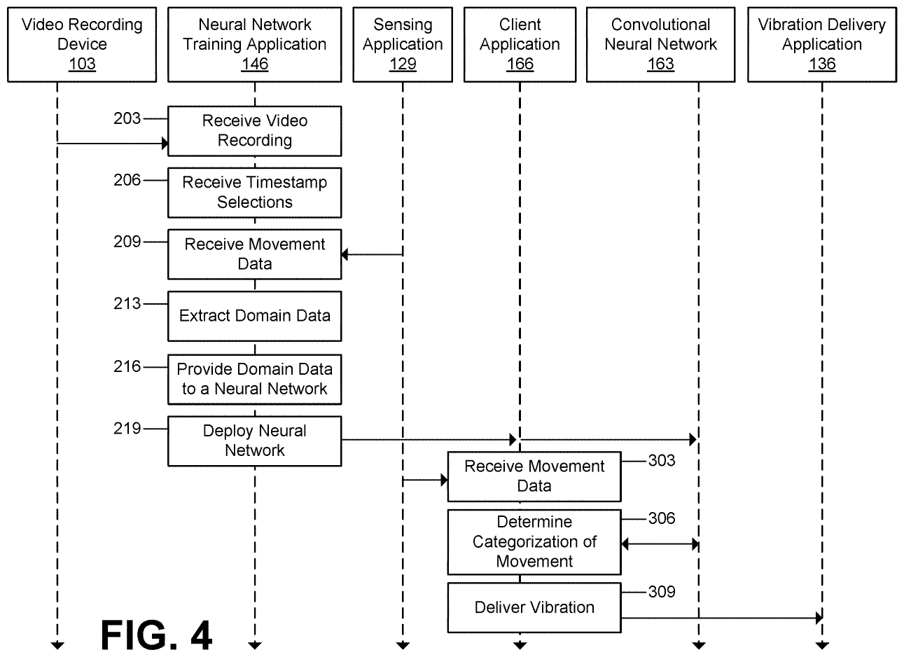

Referring next to FIG. 4, shown is a sequence diagram that illustrates the interactions between the video recording device 109, the neural network training application 146, the sensing application 129, the client application 166, CNN 163, and the vibration delivery application 136. The sequence diagram of FIG. 4 provides merely an example of the many different types of functional arrangements that can be employed to implement the operation of the depicted portion between the video recording device 109, the neural network training application 146, the sensing application 129, the client application 166, CNN 163, and the vibration delivery application 136. As an alternative, the sequence diagram of FIG. 4 can be viewed as depicting an example of elements of a method implemented in the network environment 100.

To begin, the neural network training application 146 can receive a video recording 153, as previously described in the discussion of block 203 of FIG. 2. The neural network training application 146 can receive timestamp selections and movement categorizations for the movements in the video recording 153, as previously described in the discussion of block 206 of FIG. 2. The neural network training application 146 can receive movement data 156 from the sensing application 129 of the sensing device 103, as previously described in the discussion of block 209 of FIG. 2. The neural network training application 146 can extract domain data 159, as previously described in the discussion of block 213 of FIG. 2. The neural network training application 146 can provide the domain data 159 to a neural network, as previously described in the discussion of block 216 of FIG. 2. The neural network training application 146 can be deployed on the client device 116, as previously described in the discussion of block 219 of FIG. 2.

The client application 166 can receive real-time movement data from the sensing application 129 of the sensing device 103, as previously described in the discussion of block 303 of FIG. 3. The client application 166 can determine a categorization of the movement represented in the real-time movement data, as previously described in the discussion of block 306 of FIG. 3. The client application 166 can direct the vibration delivery application 136 of the vibration delivery device 106 to provide the patient with vibration, as previously described in the discussion of block 309 of FIG. 3. After block 309, the sequence diagram of FIG. 4 ends.

A number of software components previously discussed are stored in the memory of the respective computing devices and are executable by the processor of the respective computing devices. In this respect, the term “executable” means a program file that is in a form that can ultimately be run by the processor. Examples of executable programs can be a compiled program that can be translated into machine code in a format that can be loaded into a random access portion of the memory and run by the processor, source code that can be expressed in a proper format such as object code that is capable of being loaded into a random access portion of the memory and executed by the processor, or source code that can be interpreted by another executable program to generate instructions in a random access portion of the memory to be executed by the processor. An executable program can be stored in any portion or component of the memory, including random access memory (RAM), read-only memory (ROM), hard drive, solid-state drive, Universal Serial Bus (USB) flash drive, memory card, optical disc such as compact disc (CD) or digital versatile disc (DVD), floppy disk, magnetic tape, or other memory components.

The memory includes both volatile and nonvolatile memory and data storage components. Volatile components are those that do not retain data values upon loss of power. Nonvolatile components are those that retain data upon a loss of power. Thus, the memory can include random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, or other memory components, or a combination of any two or more of these memory components. In addition, the RAM can include static random access memory (SRAM), dynamic random access memory (DRAM), or magnetic random access memory (MRAM) and other such devices. The ROM can include a programmable read-only memory (PROM), an erasable programmable read-only memory (EPROM), an electrically erasable programmable read-only memory (EEPROM), or other like memory device.

Although the applications and systems described herein can be embodied in software or code executed by general purpose hardware as discussed above, as an alternative the same can also be embodied in dedicated hardware or a combination of software/general purpose hardware and dedicated hardware. If embodied in dedicated hardware, each can be implemented as a circuit or state machine that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowcharts and sequence diagrams show the functionality and operation of an implementation of portions of the various embodiments of the present disclosure. If embodied in software, each block can represent a module, segment, or portion of code that includes program instructions to implement the specified logical function(s). The program instructions can be embodied in the form of source code that includes human-readable statements written in a programming language or machine code that includes numerical instructions recognizable by a suitable execution system such as a processor in a computer system. The machine code can be converted from the source code through various processes. For example, the machine code can be generated from the source code with a compiler prior to execution of the corresponding application. As another example, the machine code can be generated from the source code concurrently with execution with an interpreter. Other approaches can also be used. If embodied in hardware, each block can represent a circuit or a number of interconnected circuits to implement the specified logical function or functions.

Although the flowcharts and sequence diagrams show a specific order of execution, it is understood that the order of execution can differ from that which is depicted. For example, the order of execution of two or more blocks can be scrambled relative to the order shown. Also, two or more blocks shown in succession can be executed concurrently or with partial concurrence. Further, in some embodiments, one or more of the blocks shown in the flowcharts and sequence diagrams can be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein that includes software or code can be embodied in any non-transitory computer-readable medium for use by or in connection with an instruction execution system such as a processor in a computer system or other system. In this sense, the logic can include statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system. Moreover, a collection of distributed computer-readable media located across a plurality of computing devices (e.g., storage area networks or distributed or clustered filesystems or databases) may also be collectively considered as a single non-transitory computer-readable medium.

The computer-readable medium can include any one of many physical media such as magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium can be a random access memory (RAM) including static random access memory (SRAM) and dynamic random access memory (DRAM), or magnetic random access memory (MRAM). In addition, the computer-readable medium can be a read-only memory (ROM), a programmable read-only memory (PROM), an erasable programmable read-only memory (EPROM), an electrically erasable programmable read-only memory (EEPROM), or other type of memory device.

Further, any logic or application described herein can be implemented and structured in a variety of ways. For example, one or more applications described can be implemented as modules or components of a single application. Further, one or more applications described herein can be executed in shared or separate computing devices or a combination thereof. For example, a plurality of the applications described herein can execute in the same computing device, or in multiple computing devices in the same network environment 100.

Disjunctive language such as the phrase “at least one of X, Y, or Z,” unless specifically stated otherwise, is otherwise understood with the context as used in general to present that an item, term, etc., can be either X, Y, or Z, or any combination thereof (e.g., X; Y; Z; X or Y; X or Z; Y or Z; X, Y, or Z; etc.). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications can be made to the above-described embodiments without departing substantially from the spirit and principles of the disclosure. All such modifications and variations are intended to be included herein within the scope of this disclosure and protected by the following claims.

In addition to the foregoing, the various embodiments of the present disclosure include, but are not limited to, the embodiments set forth in the following clauses.

Clause 1. A method, comprising receiving, by a training application, a video recording depicting a person wearing a sensing device while walking; receiving, by the training application, a plurality of timestamp selections indicating when the person demonstrates a freezing of gait event during the video; receiving, by the training application, movement data from the sensing device associated with the video recording; extracting, by the training application, a plurality of time domains from the movement data corresponding to the plurality of timestamp selections; and sending, by the training application, the plurality of time domains to a neural network.

Clause 2. The method of clause 1, wherein the neural network is a convolutional neural network.

Clause 3. The method of clause 1 or 2, further comprising normalizing, by the training application, the plurality of time domains to make a normalized time domain; and sending, by the training application, the normalized time domain to the neural network.

Clause 4. The method of any of clauses 1, 2, or 3, further comprising generating, by the training application, a plurality of frequency domains based on the plurality of time domains; and sending, by the training application, the plurality of frequency domains to the neural network.

Clause 5. The method of clause 4, further comprising normalizing, by the training application, the plurality of frequency domains to make a normalized frequency domain; and sending, by the training application, the normalized frequency domain to the neural network.

Clause 6. The method of any of clauses 4 or 5, wherein the training application generates the plurality of frequency domains based on the plurality of time domains by performing continuous wavelet transformation on each of the plurality of time domains.

Clause 7. The method of any of clauses 1-6, wherein the movement data comprises a list of movement packets comprising gyroscopic data and accelerometer data.

Clause 8. The method of any of clauses 1-7, further comprising deploying, by the training application, the neural network on a client device.

Clause 9. A system, comprising a computing device comprising a processor and a memory; and machine-readable instructions stored in the memory that, when executed by the processor, cause the computing device to perform the method of any of clauses 1-8.

Clause 10. A non-transitory, computer-readable medium, comprising machine-readable instructions that, when executed by a processor, cause a computing device to perform the method of any of clauses 1-8.

Clause 11. A method, comprising receiving, by a client application, movement data from a sensing device for a movement by a person; determining, by the client application, a categorization of the movement by processing the movement data through a convolutional neural network; and in response to determining that the categorization of the movement is indicative of freezing of gait, directing, by the client application, a vibration delivery device to deliver vibration.

Clause 12. The method of clause 11, wherein the sensing device comprises a 3-axis accelerometer and a 3-axis gyroscope.

Clause 13. The method of clause 11 or 12, wherein the convolutional neural network classifies the movement as an unintentional stop which is indicative of freezing of gait.

Clause 14. The method of clause 11 or 12, wherein the convolutional neural network classifies the movement as abnormal walking pattern indicative of freezing of gait.

Clause 15. The method of any of clauses 11-14, wherein the client application directs the vibration delivery device to deliver vibration at a frequency between 5 HZ and 300 HZ.

Clause 16. The method of any of clauses 11-15, wherein the client application directs the vibration delivery device to deliver vibration at an amplitude less than 0.8 millimeters.

Clause 17. The method of any of clauses 11-14, wherein the client application directs the vibration delivery device to deliver vibration in a sustained frequency and a sustained amplitude between 0.5 seconds and 5 seconds.

Clause 18. The method of any of clauses 11-14, wherein the client application directs the vibration delivery device to deliver vibration in a variable frequency and a variable amplitude between 0.5 seconds and 5 seconds.

Clause 19. The method of at least one of clauses 11-14, wherein the client application directs the vibration delivery device to deliver vibration in a sustained frequency and a variable amplitude between 0.5 seconds and 5 seconds.

Clause 20. The method of at least one of clauses 11-14, wherein the client application directs the vibration delivery device to deliver vibration in a variable frequency and a variable amplitude between 0.5 seconds and 5 seconds.

Clause 21. The method of at least one of clauses 11-16, wherein the client application directs the vibration delivery device to deliver vibration in a pulsing pattern.

Clause 22. A system, comprising a sensing device; a vibration delivery device; a computing device comprising a processor and a memory; and machine-readable instructions stored in the memory that, when executed by the processor, cause the computing device to perform the method of any of clauses 11-21.

Clause 23. A non-transitory, computer-readable medium, comprising machine-readable instructions that, when executed by a processor, cause a computing device to perform the method of any of clauses 11-21.

1. A method, comprising:
receiving, by a training application, a video recording depicting a person wearing a sensing device while walking;
receiving, by the training application, a plurality of timestamp selections indicating when the person demonstrates a freezing of gait event during the video;
receiving, by the training application, movement data from the sensing device associated with the video recording;
extracting, by the training application, a plurality of time domains from the movement data corresponding to the plurality of timestamp selections; and
sending, by the training application, the plurality of time domains to a neural network.

2. The method of claim 1, wherein the neural network is a convolutional neural network.

3. The method of claim 1, further comprising:
normalizing, by the training application, the plurality of time domains to make a normalized time domain; and
sending, by the training application, the normalized time domain to the neural network.

4. The method of claim 1, further comprising:
generating, by the training application, a plurality of frequency domains based on the plurality of time domains; and
sending, by the training application, the plurality of frequency domains to the neural network.

5. The method of claim 4, further comprising:
normalizing, by the training application, the plurality of frequency domains to make a normalized frequency domain; and
sending, by the training application, the normalized frequency domain to the neural network.

6. The method of claim 4, wherein the training application generates the plurality of frequency domains based on the plurality of time domains by performing continuous wavelet transformation on each of the plurality of time domains.

7. The method of claim 1, wherein the movement data comprises a list of movement packets comprising gyroscopic data and accelerometer data.

8. The method of claim 1, further comprising deploying, by the training application, the neural network on a client device.

9. (canceled)

10. (canceled)

11. A method, comprising:
receiving, by a client application, movement data from a sensing device for a movement by a person;
determining, by the client application, a categorization of the movement by processing the movement data through a convolutional neural network; and
in response to determining that the categorization of the movement is indicative of freezing of gait, directing, by the client application, a vibration delivery device to deliver vibration.

12. The method of claim 11, wherein the sensing device comprises a 3-axis accelerometer and a 3-axis gyroscope.

13. The method of claim 11, wherein the convolutional neural network classifies the movement as an unintentional stop which is indicative of freezing of gait.

14. The method of claim 11, wherein the convolutional neural network classifies the movement as abnormal walking pattern indicative of freezing of gait.

15. The method of claim 11, wherein the client application directs the vibration delivery device to deliver vibration at a frequency between 5 HZ and 300 HZ.

16. The method of claim 11, wherein the client application directs the vibration delivery device to deliver vibration at an amplitude less than 0.8 millimeters.

17. The method of claim 11, wherein the client application directs the vibration delivery device to deliver vibration in a sustained frequency and a sustained amplitude between 0.5 seconds and 5 seconds.

18. The method of claim 11, wherein the client application directs the vibration delivery device to deliver vibration in a variable frequency and a variable amplitude between 0.5 seconds and 5 seconds.

19. The method of claim 11, wherein the client application directs the vibration delivery device to deliver vibration in a sustained frequency and a variable amplitude between 0.5 seconds and 5 seconds.

20. The method of claim 11, wherein the client application directs the vibration delivery device to deliver vibration in a variable frequency and a variable amplitude between 0.5 seconds and 5 seconds.

21. The method of claim 11, wherein the client application directs the vibration delivery device to deliver vibration in a pulsing pattern.

22. A system, comprising:
a sensing device;
a vibration delivery device;
a computing device comprising a processor and a memory; and
machine-readable instructions stored in the memory that, when executed by the processor, cause the computing device to at least:
receive movement data from a sensing device for a movement by a person;
determine a categorization of the movement by processing the movement data through a convolutional neural network; and
in response to a determination that the categorization of the movement is indicative of freezing of gait, direct the vibration delivery device to deliver vibration.

23. (canceled)
